(19) United States
(12) Patent Application Publication
(10) Pub. No.: US 2023/0301959 A1
(43) Pub. Date: Sep. 28, 2023

(54) COMBINATIONS OF CARNOSINE AND ZINC FOR THE TREATMENT AND PREVENTION OF VIRAL INFECTIONS

(71) Applicant: Deanna J. Nelson, Raleigh, NC (US)

(72) Inventor: Deanna J. Nelson, Raleigh, NC (US)

(21) Appl. No.: 18/090,659

(22) PCT Filed: Jun. 23, 2021
(86) PCT No.: PCT/US2021/038706

§ 371 Date: Dec. 29, 2022

Related U.S. Application Data
(60) Provisional application No. 63/046,846, filed on Jul. 1, 2020.

(51) Int. Cl. A61K 31/315

(52) U.S. Cl. CPC A61K 31/315

(57) ABSTRACT

The present invention relates to the treatment of viral infection, and in particular to the use of an amount of a chelate of zinc and L-camosineeffective to treat or to prevent infections associated with viruses in said human cells.

### CROSS-REFERENCE TO RELATED APPLICATIONS

This application claims the benefit of U.S. Prov. Appl. 63/046,846 filed Jul. 1, 2020, the entire contents of which are incorporated herein by reference.

### FIELD OF THE INVENTION

The present invention relates to the treatment of viral infection, and in particular to the use of an amount of carnosine and zinc effective to treat or to prevent infections associated with viruses in said human cells.

### BACKGROUND OF THE INVENTION

Viruses of widely divergent type present a major health hazard for humans, animals and plants and can result in harmless to highly dangerous infections. In contrast to bacteria, viruses require a living host cell for life, development of their activity, and for propagation.

Recent pandemics, such as those associated with corona viruses exemplified by SARS-CoV-19 and influenza viruses such as H1N1, illustrate the impact that viruses have on societies around the globe. At the present time, no satisfactory wide-ranging antiviral drug for any or all of the multiple strains of SARS-CoV-19 is available, with the possible exception of remdesivir, which is being tested clinically.

Prevention is particularly desirable in cases involving corona or influenza viruses. Recent data indicate that the risk of exposure is high and the probability of subjects manifesting highly dangerous infections and mortality is significant.

All of the remedies proposed to date were directed toward a direct combatting of the virus after its entrance into the cell, in order to inactivate or kill it there.

What is needed in the art are agents and methods that can be used to prevent viral infection, especially by single stranded RNA viruses such as SARS-CoV-19, in subjects in need thereof.

### SUMMARY OF THE INVENTION

The present invention relates to the treatment of viral infection, and in particular to the use of an amount of carnosine and zinc ion effective to treat or to prevent infections associated with viruses in said human cells.

In some preferred embodiments, the present invention provides methods for preventing, inhibiting or treating viral infections or their physiological immune-related sequellae in cases involving single-stranded RNA viruses in a subject in need thereof comprising administering to the subject an amount of carnosine and zinc ion effective to treat or to prevent disorders associated with infections of said viruses in cells in said subject. In some preferred embodiments, the carnosine and a zinc are provided as an L-carnosine-zinc chelate. In some preferred embodiments, the subject is a human subject. In some preferred embodiments, the single-stranded RNA virus is a coronavirus. In some preferred embodiments, the coronavirus is a SARS-CoV virus. In some preferred embodiments, the SARS-CoV virus is SARS-CoV-2.

In some preferred embodiments, the present invention provides carnosine and zinc ion for use in preventing, inhibiting or treating viral infection by single stranded RNA viruses in the cells of a mammalian subject. In some preferred embodiments, the carnosine and zinc ion are first admixed with a carrier in the amount of 1 mg to 41 mg zinc L-carnosine chelate per milliliter or per gram of carrier. In some preferred embodiments, the carnosine and zinc ion are formulated with a carrier to provide from 2 to 800 micromoles zinc per milliliter or gram of carrier. In some preferred embodiments, the subject is a human subject. In some preferred embodiments, the single-stranded RNA virus is a coronavirus. In some preferred embodiments, the coronavirus is a SARS-CoV virus. In some preferred embodiments, the SARS-CoV virus is SARS-CoV-2.

In some preferred embodiments, the present invention provides kits comprising a first container comprising an effective dose of zinc L-carnosine chelate and a second container comprising a sterile diluent. In some preferred embodiments, the diluent is an aqueous diluent. In some preferred embodiments, the kits are provided for use in preventing, inhibiting or treating viral infection by single-stranded RNA viruses in the cells of a mammalian subject. In some preferred embodiments, the subject is a human subject. In some preferred embodiments, the single-stranded RNA virus is a coronavirus. In some preferred embodiments, the coronavirus is a SARS-CoV virus. In some preferred embodiments, the SARS-CoV virus is SARS-CoV-2.

### DETAILED DESCRIPTION OF THE INVENTION

The present disclosure relates generally to medical treatments. More specifically, the present disclosure relates to therapeutic solutions suitable for either oral or intravenous therapy. In some preferred embodiments, the present invention relates to the treatment of viral infection, and in particular to the use of an amount of carnosine and a second amount of zinc effective to treat or to prevent infections associated with viruses in said human cells. Surprisingly, a remedy has now been developed that acts not only directly on virus replication, but also inactivates the virus by means of a still unexplained change in the host cell and severely inhibits virus propagation and survival.

In some preferred embodiments, a remedy of the invention provides continued benefit during recovery and convalescence, when the risk of manifestation of physiological after-effects of virus exposure continues. Although it is too early to state with certainty, the extent of RNAnemia and the severity of COVID-related disease suggest that during recovery and convalescence, SARS-CoV-2 virus may persist in both body tissues and fluids such as saliva, tears, sweat, urine, semen, and cervical secretions. The virus may also remain viable for extended periods on various exposed surfaces and in deposits of dried biological materials. If so, it is highly likely that immune-mediated sequelae similar to other viral ailments will be manifested.

Despite the risk for exaggeration, an examination of the pathological manifestations of post-Ebola virus disease syndrome (PEVDS) may be useful for planning for post-SARS-CoV-2 recovery and convalescence support. Manifestations of PEVDS include polyarthralgias, abdominal pain, alopecia, anorexia, fatigue, fever, headaches, sleep disturbances, uveitis, peripheral dysethesias, short-term memory problems, erectile dysfunctions, lethargy, and mood disorders.

It is too early to predict whether SARS-Cov-2 virus patients will exhibit immune-mediated sequelae similar to those manifested after other viral ailments. Nonetheless, early reports suggest that loss of taste and/or smell, musculoskeletal pain, abdominal pain, neurological disorders (e.g., short-term memory loss and sleep disorders), and depression may characterize post-recovery pathologies associated with SARS-CoV-2 infection.

In some preferred embodiments, the present invention provides a method process for preventing or treating viral infections or their physiological immune-related sequellae in cases involving single-stranded RNA viruses in human cells comprising administering an amount of carnosine and zinc effective to treat or to prevent infections associated with the viruses in said human cells. In some embodiments the agents for treating virus infections and preventing virus-infection related disorders are preferably provided in the preparation form of a mouth wash, intraoral ointment, or lozenge. In some preferred embodiments, the agents of the present invention are embodied in a zinc L-carnitine chelate.

Accordingly, the present disclosure relates generally to combinations of zinc and carnosine and methods of making and using formulations of same. More specifically, the present disclosure relates to solutions comprising a stable and therapeutically effective amount of zinc L-carnosine chelate. The solutions can be prepared, for example, in a diluent suitable for oral administration. Alternatively, the solutions can be prepared in a sterile diluent or in a diluent that is subjected to filtration as a means of sterilization immediately before intravenous administration. In some preferred embodiments, the zinc L-carnosine chelate is admixed with a carrier in the amount of 1 mg to 41 mg zinc L-carnosine chelate per milliliter or per gram of carrier. In some preferred embodiments, the zinc L-carnosine chelate is admixed with a carrier in the amount of 10 micrograms zinc to 800 micrograms zinc per dose. In some preferred embodiments, the carrier is a pharmaceutically acceptable carrier suitable for parenteral administration. In some preferred embodiments, the carrier is a sterile aqueous diluent.

L-Carnosine is a dipeptide, β-alanyl-L-histidine. L-Carnosine occurs naturally in the skeletal system, muscles, and brain of vertebrates. As an antioxidant, anti-aging, antihypertensive, antineoplastic agent, and buffer, it has been shown to play a protective role in wound healing, immune function, diabetes, and loss of vision as a result of cataract formation. Carnosine acts as a chelating agent to reduce levels of heavy metals in the bloodstream.

Carnosine has been proposed as a supplement that could be useful in management of COVID-19. [Jindal et al., The prevention and management of COVID-19: Seeking a practical and timely solution. (2020) Int’l J. Envir, Res. Publ. Health 17, 3986.] A common feature of all virus-induced human diseases is the sustained increase in levels of iNOS and Nitric Oxide (NO). [Perrone, L.A.; Belser, J.A.; Wadford, D.A.; Katz, J.M.; Tumpey, T.M. Inducible nitric oxide contributes to viral pathogenesis following highly pathogenic influenza virus infection in mice. J. Infect. Dis. 2013, 207, 1576-1584; Majano, P.L.; García-Monzón, C.; López-Cabrera, M.; Lara-Pezzi, E.; Fernández-Ruiz, E.; Garcia-Iglesias, C.; Borque, M.J.; Moreno-Otero, R. Inducible nitric oxide synthase expression in chronic viral hepatitis. Evidence for a virus-induced gene upregulation. J. Clin. Investig. 1998, 101, 1343-1352.] While increased NO concentrations are protective against microbial infections, the opposite is true in the case of viral infections. [James, S.L. Role of nitric oxide in parasitic infections. Microbiol. Rev. 1995, 59, 533-547.] In the latter scenario, NO reacts with oxygen free radicals to produce highly reactive peroxynitrites, which in turn damage tissue and DNA through the nitrosylation of cellular proteins and molecules. [Akaike, T. Role of free radicals in viral pathogenesis and mutation. Rev. Med. Virol. 2001, 11, 87-101; Akaike, T.; Noguchi, Y.; Ijiri, S.; Setoguchi, K.; Suga, M.; Zheng, Y.M.; Dietzschold, B.; Maeda, H. Pathogenesis of influenza virus-induced pneumonia: Involvement of both nitric oxide and oxygen radicals. Proc. Natl. Acad. Sci. USA 1996, 93, 2448-2453; Akaike, T.; Maeda, H. Nitric oxide and virus infection. Immunology 2000, 101, 300-308. Owing to its anti-inflammatory and antioxidant properties, carnosine can reduce the concentration of highly reactive peroxynitrites in the human body, aiding the immune fight against viral infections such as influenza A, dengue fever and Zika. Babizhayev, M.A.; Deyev, A.I. Management of the virulent influenza virus infection by oral formulation of nonhydrolized carnosine and isopeptide of carnosine attenuating proinflammatory cytokine-induced nitric oxide production. Am. J. Ther. 2012, 19, e25-e47.] Furthermore, in liver cell culture assays, carnosine has been shown to significantly inhibit viral genome replication and to ameliorate cell viability post infection. [Rothan, H.A.; Abdulrahman, A.Y.; Khazali, A.S.; Nor Rashid, N.; Chong, T.T.; Yusof, R. Carnosine exhibits significant antiviral activity against Dengue and Zika virus. J. Pept. Sci. 2019, 25, e3196.]

In a laboratory study of BALB/c female mice infected withH9N2 influenza virus, 7 consecutive days of carnosine administered orally (10 mg/kg body mass) significantly reduced levels of TNF-a, IL-1b, TLR-4 mRNA and protein, as well as decreasing overall mortality (43% versus 75%, P < 0.05). An improvement in pathological lung lesions, decreased lung wet mass ratio, and reduced myeloperoxidase activity also was reported. We similarly hypothesize that the oral administration of carnosine may play an important role in reducing the lung tissue damage associated with SARS-CoV-2 infection and hence associated morbidity and mortality. [de Courten, B.; Jakubova, M.; de Courten, M.P.; Kukurova, I.J.; Vallova, S.; Krumpolec, P.; Valkovic, L.; Kurdiova, T.; Garzon, D.; Barbaresi, S.; et al. Effects of carnosine supplementation on glucose metabolism: Pilot clinical trial. Obes. (Silver Spring) 2016, 24, 1027-1034; Baraniuk, J.N.; El-Amin, S.; Corey, R.; Rayhan, R.; Timbol, C. Carnosine treatment for Gulf War illness: A randomized controlled trial. Glob. J. Health Sci. 2013, 5, 69-81.]

Genetic variants in Apolipoprotein E (ApoE), which are involved in regulatory checkpoint processes of the innate immune system and associated antigen-antibody complexes, also may underlie the therapeutic effects of carnosine, autonomous of its antiviral properties. [Noris, M.; Remuzzi, G. Overview of complement activation and regulation. Semin. Nephrol. 2013, 33, 479-492; Vignesh, P.; Rawat, A.; Sharma, M.; Singh, S. Complement in autoimmune diseases. Clin. Chim. Acta 2017, 465, 123-130; Sellar, G.C.; Blake, D.J.; Reid, K.B. Characterization and organization of the genes encoding the A-, B-and C-chains of human complement subcomponent C1q. The complete derived amino acid sequence of human C1q. Biochem. J. 1991, 274, 481-490.] Compared with ApoE e3e3 homozygotes, COVID-19 positivity occurs more frequently among e4e4 homozygotes (OR = 2.3, 95%CI = 1.7-3.2), with increased severity being independent of pre-existing dementia, cardiovascular disease, and type-2 diabetes. [Kuo, C.L.; Pilling, L.C.; Atkins, J.L.; Masoli, J.A.H.; Delgado, J.; Kuchel, G.A.; Melzer, D. APOE e4 genotype predicts severe COVID-19 in the UK Biobank community cohort. J. Gerontol. Ser. A Biol. Sci. Med. Sci. 2020.] Both ACE2 and ApoE are highly co-expressed genes in type II alveolar cells in the lungs. [Zhao, Y.; Zhao, Z.; Wang, Y.; Zhou, Y.; Ma, Y.; Zuo, W. Single-cell RNA expression profiling of ACE2, the receptor of SARS-CoV-2. BioRxiv 2020.] Among ApoE4 positive carriers, carnosine supplementation conveys positive benefits for mild cognitive impairment and blood flow in the prefrontal cortex of the brain. [Masuoka, N.; Yoshimine, C.; Hori, M.; Tanaka, M.; Asada, T.; Abe, K.; Hisatsune, T. Effects of Anserine/Carnosine supplementation on mild cognitive impairment with APOE4. Nutrients 2019, 11; Ding, Q.; Tanigawa, K.; Kaneko, J.; Totsuka, M.; Katakura, Y.; Imabayashi, E.; Matsuda, H.; Hisatsune, T. Anserine/Carnosine supplementation preserves blood flow in the prefrontal brain of elderly people carrying APOE e4. Aging Dis. 2018, 9, 334-345. Dietary administration of carnosine also has been shown to prevent early atherosclerotic lesion formation in ApoE-null mice, as well as attenuating renal disease. Barski, O.A.; Xie, Z.; Baba, S.P.; Sithu, S.D.; Agarwal, A.; Cai, J.; Bhatnagar, A.; Srivastava, S. Dietary carnosine prevents early atherosclerotic lesion formation in apolipoprotein E-null mice. Arterioscler. Thromb. Vasc. Biol. 2013, 33, 1162-1170; Menini, S.; Iacobini, C.; Ricci, C.; Scipioni, A.; Blasetti Fantauzzi, C.; Giaccari, A.; Salomone, E.; Canevotti, R.; Lapolla, A.; Orioli, M.; et al. D-Carnosine octylester attenuates atherosclerosis and renal disease in ApoE null mice fed aWestern diet through reduction of carbonyl stress and inflammation. Br. J. Pharmacol. 2012, 166, 1344-1356.]

The regular administration of carnosine (in combination with forskolin, homotaurine, vitamins B1, B2, and B6, folic acid, and magnesium) for 2-4 months, has been shown to be safe for humans of different age groups. In two separate studies, one in obese patients with Type-II diabetes and the other in Gulf-War Veterans, the use of carnosine for 2 weeks was well tolerated without any reported adverse reactions.

L-Carnosine is one of several agents that can form stable chelates with zinc. Other chelating agents useful for zinc complexation include L-histidine, citrate, penicillamine, L-anserine, L-cysteine, penicillanic acid, and 2-picolinic acid. As a result of chelation, the health benefits of both the zinc and the chelating agent are enhanced. Zinc chelation increases absorption and membrane permeability of zinc and enables concurrent delivery of both zinc and the chelating agent(s) to the systemic circulation or to the cytosol of a cell.

The zinc L-carnosine useful in the practice of the present invention may be either amorphous or crystalline. There is no difference between the amorphous and crystalline forms of zinc L-carnosine in the therapeutic and prophylactic effects on zinc-sensitive viruses and disorders related thereto. Crystalline zinc L-carnosine and amorphous zinc L-carnosine can be prepared in accordance with the processes described in Japanese Patent Publication No. 115150/1995 and Japanese Patent Publication No. 5367/1991, respectively (both of which are incorporated herein by reference in their entirety). A production process for preparation of crystalline L-carnosine zinc complex is also described by Hirano and Katayama in U.S. Patent No. 6,169,083, incorporated herein by reference in its entirety. Zinc L-carnosine is commercially available as a compound named polaprezinc.

A variety of uses of zinc L-carnosine as either a drug or a dietary supplement are known. Pharmaceutical formulations of zinc L-carnosine are used in Japan and Korea as treatments for peptic ulcers and H. pylori infections. In North America zinc L-carnosine is recognized by the U.S. Food and Drug Administration as a New Dietary Ingredient suitable for use in dietary and nutritional supplements. Supplements containing zinc L-carnosine are administered by mouth for a variety of disorders including gastrointestinal disorders, inflammatory bowel diseases, osteoporosis, taste disorders, skin lesions, liver dysfunction, and oral mucositis resulting from chemo- or radiotherapy.

The zinc ion (Zn²⁺) plays an essential role in supporting growth and development and maintaining a healthy immune system. Although knowledge about zinc homeostasis in mammals continues to evolve, the ion is recognized as a key structural component in about 10% of the human proteome and participates in numerous cellular functions including, but not limited to, cell proliferation and differentiation, RNA and DNA synthesis, stabilization of cell structures /membranes, as well as redox regulation and apoptosis. Moreover, strong correlations have been established between zinc deficiency and metabolic and chronic diseases, including type 1 diabetes, rheumatoid arthritis, cancer, neurodegenerative diseases, osteoporosis, and depression, as well as the incidence and severity of infectious diseases such as shigellosis, acute cutaneous leishmaniosis, malaria, human immunodeficiency virus, tuberculosis, measles, and pneumonia.

Zinc deficiency is strikingly common, and current estimates indicate that over two billion people worldwide exhibit deficiency in this metal. Zinc deficiency has been identified as the 5^{th} leading life-threatening factor in developing countries. Likewise, in industrialized nations, zinc deficiency is commonly found in people with gastrointestinal disorders reducing mineral uptake, including reductions related to age, lifestyle, and the use of medications and supplements that alter mineral uptake. Consequently, zinc status is a critical factor that can influence antiviral immunity, particularly as zinc-deficient populations are often most at risk of acquiring viral infections. Moreover, low plasma zinc concentrations are associated with increased numbers of organ failures and measures of inflammation in severe disease.

In general, therefore, zinc supplementation has been administered to mitigate the effects of injury and illness and the associated dysregulation of the immune system that result in significant morbidity and mortality. Both oral and intravenous zinc supplementation have proven useful in replenishing deficiencies in plasma and whole-body zinc levels.

However, nothing was known about the fact that combinations of L-carnosine and zinc exhibit therapeutic and prophylactic effects on some viruses and virus-related disorders, including disorders related to single-stranded RNA viruses such as coronaviruses.

Historical in vitro studies with purified rhinovirus and poliovirus 3C proteases revealed that protease activity was inhibited by zinc ion. Zinc ions also inhibited polyprotein processing in cells infected with human rhinovirus and coxsackievirus B3. Moreover, an inhibitory effect of zinc ion on the activity of viral RNA-dependent RNA polymerase (RdRp) from rhinoviruses and hepatitis C was noted. This is a particularly significant finding, since RdRp is the core enzyme of the viral multiprotein replication and transcription complex. In 2010, te Velthuis and coworkers reported that the zinc ion (Zn2+) inhibits both coronavirus and arterivirus RNA polymerase activity in vitro and zinc ionophores block the replication of these viruses in cell culture. [te Velthuis AJW, et al. PLoS Pathogens 2010; 6(11), e1001176.] This finding is particularly significant, since the plethora of diseases related to SARS-CoV-2 lack an effective prophylactic or treatment.

Despite the promise that the zinc ion might reduce the activity and integrity of some viruses, zinc’s antiviral activity is dependent on its transport from the external environment into the cell. Unless this movement is facilitated by a zinc ionophore, high and potentially toxic concentrations of water-soluble zinc salts are required to enable antiviral activity. The utility of known zinc ionophores such as hinokitol, pyrrolidine dithiocarbamate, and both pyrithione and its derivatives that are described in U.S. Pat. 8,507,531 to Magda et al. is severely restricted by the human toxicity of these ionophores.

Unexpectedly, Applicant has discovered that combinations of carnosine and zinc ion exhibit synergistic antiviral activity through multiple independent mechanisms of viral inactivation. Preferably the combinations of L-carnosine and zinc ion are complexes of either crystalline or amorphous zinc L-carnosine. Alternatively, the combinations of L-carnosine and zinc may also contain a second zinc ionophore such as L-histidine, L-anserine, L-cysteine, penicillamine, or 2-picolinic acid.

It is contemplated that after administration of a combination of the invention for the treatment of a virus-related disorder, each component continues to provide therapeutic benefits that have been reported in the past. Thus, L-carnosine continues to exhibit the pleiotropic activities reported by Prokopieva et al., for example, but also serves as a zinc ionophore by facilitating transport of the zinc ion into infected cells. As a result, combinations of carnosine and zinc ion exhibit potent antiviral activity at zinc concentrations ranging from about 2 micromolar to about 50 micromolar zinc ion, concentrations of zinc ion which have little or no cellular toxicity.

The present invention derives in part from analysis of published reports of the mechanisms of activation and cell entry of the coronavirus named SARS-CoV-2. SARS-CoV-2 is an enveloped, non-segmented, positive sense RNA virus that is included in the sarbecovirus, orthocoronaviridae subfamily which is broadly distributed in humans and other mammals. Within its envelope the virus contains single strands of RNA as well as heavily phosphorylated N-protein that enables its replication and propagation. The outer surface of the virus is characterized by crown-like glycoprotein spikes. Two other structural glycoproteins, small envelope glycoprotein and membrane glycoprotein, are required to maintain structural integrity as well as promotion of replication and propagation within the host cell.

The spike glycoprotein is a transmembrane protein made up of homotrimers protruding in the viral surface. This glycosylated protein is the “silver bullet” that associates with and binds to host cells with angiotensin-converting enzyme-2 (ACE2) expressed on their surface. A recent report by Shang et al. suggests that the binding elements of the spike glycoprotein are “hidden,” a feature that reduces the likelihood of early detection by the immune system. The binding elements are exposed after activation by furin, a serine protease that is found throughout the body.

Furin is richly expressed in the mouth. Likewise, ACE2 is highly expressed in the tongue, oral cavity, upper esophagus and stratified epithelial cells, as well as type II alveolar cells of the lungs. In addition, ACE2 is highly expressed throughout the body, on cells found in the ileum and colon, cholangiocytes, myocardial cells, kidney proximal tubule cells, and bladder urothelial cells. Therefore, patients who are infected with this virus experience respiratory problems ranging from persistent coughing to Acute Respiratory Distress Syndrome, but also may experience disorders of the heart, circulatory system, kidneys, and digestive tract.

The disease process begins through the attachment of S-glycoprotein to the ACE2 receptor. Attachment occurs in the binding region of S-protein ligands found at residues 331 to 524 of the S-protein. Entry and binding are then followed by fusion of the viral membrane and the host cell.

Once integrated into the host cell, the virus rapidly propagates. Within a week after infection, a symptomatic patient may have 5-15 x 10¹⁰ copies of the virus per milliliter of saliva in the deep throat. Both sputum and broncholavage fluid likely contain high titers of virus, blooms of bacteria, and cell debris.

Thus, Applicant contemplates that a novel composition of the invention exerts multiple actions following its administration. First, it enhances the bioavailability and membrane permeability of the zinc ion, reducing the concentration of zinc that must be administered to inhibit both furin (the serine protease required to activate the spike protein on the virus) and the virus itself. Furin inhibition also allows the innate immune system to be more effective, since furin activates cytokines and other inflammatory factors. Second, the zinc ion serves as a Trojan horse, binding to the spike protein on the virus and inhibiting the ability of the spike protein to associate with zinc ions on the ACE2 receptor (the preferred virus binding site). Third, the zinc ion inactivates the viral proteins that are required for propagation within the host cell. This action allows the innate immune response to be more effective. Fourth, the composition of the invention provides supplemental zinc to the subject, beneficially replenishing the supply of zinc throughout the body. Fifth, a composition of the invention acts to ameliorate viral-related disorders, such as loss of taste, vascular inflammation, muscle pain and dysfunction, inflammation in the lung, kidney, liver, and intestines, disorders of cognition, and other clinical manifestations of exposure to a virus such as SARS-CoV-2.

At the same time, a composition of the invention provides L-carnosine, an endogenous peptide that is an antioxidant and antiglycating agent.

Briefly summarized, it is contemplated that the agents which compose a composition of the invention inhibit virus activation by furin, inhibit viral propagation within the host cell, replenish the supply of zinc within the host’s body, reduce the intensity of the cytokine storm and systemic inflammation that characterizes COVID-19, and restore the sense of taste that has been lost as a result of viral infection.

If a combination of the invention is administered orally, the recommended dosage for adult men and women is 12 mg Zn/day and 9 mg Zn/day, respectively. Absorption from the gastrointestinal tract into the systemic circulation ranges from 2-16% of the administered dose.

If a combination of the invention is administered parenterally, doses totalling 100-500 microgram Zn/kg/d are well tolerated. Each dose may be divided into parts for administration at several times during the day.

Accordingly, while in some embodiments it is possible for combinations of L-carnosine and zinc ion to be administered separately, it is often preferable to administer it as a composition or formulation. In some preferred embodiments, the composition is a pharmaceutical composition (e.g., formulation, preparation, medicament) comprising zinc L-carnosine, as described herein, and a pharmaceutically acceptable carrier, diluent, or excipient.

Pharmaceutical and nutraceutical compositions of the present invention preferably comprise an effective amount of a chelate of zinc and L-carnosine (“chelate” or “chelate composition”).

In preferred embodiments of the present invention, the chelate composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In some preferred embodiments, the composition is a pharmaceutical composition comprising at least one carnosine-zinc complex, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilizers, solubilizers, surfactants (e.g., wetting agents), masking agents, coloring agents, flavoring agents, and sweetening agents. In some preferred embodiments, the O-acyl L-carnitine may be provided as kit with the O-acyl L-carnitine in one container and one or more of a suitable pharmaceutically acceptable carrier, diluent, excipient, adjuvant, filler, buffer, preservative, anti-oxidant, lubricants, stabilizers, solubilizers, surfactants (e.g., wetting agents) in one or more additional containers.

In some preferred embodiments, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M, Ash and I. Ash), 2001 (Synapse Information Resources, Inc., Endicott, N.Y., USA), Remington’s Pharmaceutical Sciences, 20th edition, pub. Lippincott, Williams & Wilkins, 2000; and Handbook of Pharmaceutical Excipients, 2nd edition, 1994.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer’s Solution, or Lactated Ringer’s Injection. Extemporaneous injection solutions and suspensions may be prepared from sterile powders, granules, and tablets.

For oral administration the chelate compositions of the present invention may alternatively be incorporated with one or more excipients in the form of a mouthwash (including suspensions, emulsions and syrups), dentifrice, buccal tablet, oral spray, or sublingual orally-administered formulation. For example, a mouthwash may be prepared incorporating the active ingredient in the required amount in an appropriate solvent, such as a sodium borate solution (Dobell’s Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth. Mouth wash or intraoral rinse embodiments of the invention can be formulated by suitably selecting in combination pharmaceutically acceptable additives from, for example, distilled water for injection, purified water, sodium carboxymethyl cellulose, lactose, sorbitol, mannitol, xylitol, lactitol, gum Arabic, tragacanth gum, gelatin, glycerol, polyoxyethylene derivatives, etc.

In some embodiments of the present invention, the composition is combined or mixed thoroughly with a semi-solid or solid carrier. In some preferred embodiments, the lipid compositions are incorporated into chewable matrices. Preferred chewable matrices jelly candies and gelatin-based gummi candy. Exemplary gummi candies include gummi bears, gummi worms, gummi frogs, gummi hamburgers, gummi cherries, gummi soda bottles, gummi sharks, gummi army men, gummi hippopotami, gummi lobsters, gummi watermelons, gummi octopuses, gummi apples, gummi peaches, and gummi oranges. The terms “gummi” and “gummy” are used interchangeably herein.

In other preferred embodiments of the invention, the active compound may be formulated for administration via other routes, for example, topical (i.e., transdermal) administration, mucosal administration (intranasal, vaginal, etc.) and/or inhalation.

In some embodiments, the chelate compositions are formulated for oral administration with flavoring agents or sweeteners. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate. Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. In addition to the carbohydrates described above, the nutritional supplement can contain natural or artificial (preferably low calorie) sweeteners, e.g., saccharides, cyclamates, aspartamine, aspartame, acesulfame K, and/or sorbitol.

The chelate compositions of the present invention may also be delivered as nutraceuticals, dietary supplements, nutritional supplements, or functional foods.

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: ascorbates (ascorbic acid, mineral ascorbate salts, rose hips, acerola, and the like), dehydroepiandosterone (DHEA), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In other embodiments, the present invention provides nutritional supplements (e.g., energy bars or meal replacement bars or beverages) comprising of the chelate compositions of the present invention. In preferred embodiments, the nutritional supplements comprise an effective amount of the components as described above. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked comstarch).

### REFERENCES CITED

1. Braun E, Hotter D, Koepke L, et al. Guanylate-Binding Proteins 2 and 5 Exert Broad Antiviral Activity by Inhibiting Furin-Mediated Processing of Viral Envelope Proteins. Cell Rep. 2019; 27(7): 2092-2104.e10.

2. Braun E, Sauter D. Furin-mediated protein processing in infectious diseases and cancer. Clin Transl Immunology. 2019; 8(8): e1073. Published 2019 Aug 5. doi:10.1002/cti2.1073.

3. Funato M, Ozeki M, Suzuki A, Ishihara M, Kobayashi R, Nozawa A, Yasue S, Endo-Ohnishi S, Fukao T, Itoh Y. Prophylactic Effect of Polaprezinc, a Zinc-L-carnosine, Against Chemotherapy-induced Oral Mucositis in Pediatric Patients Undergoing Autologous Stem Cell Transplantation. Anticancer Res. 2018 Aug; 38(8): 4691-4697.

4. Furuta S, Toyama S, Sano H. Absorption mechanism of polaprezinc (zinc L-carnosine complex) by an everted sac method. Xenobiotica. 1994; 24(11): 1085-1094.

5. Ghaffari H, Tavakoli A, Moradi A, Tabarraei A, Bokharaei-Salim F, Zahmatkeshan M, Farahmand M, Javanmard D, Kiani SJ, Esghaei M, Pirhajati-Mahabadi V, Monavari SH, Ataei-Pirkooh A. Inhibition of H1N1 influenza virus infection by zinc oxide nanoparticles: another emerging application of nanomedicine. J Biomed Sci. 2019 Sep 10; 26(1): 70. doi: 10.1186/s12929-019-0563-4.

6. Hayashi H, Kobayashi R, Suzuki A, et al. Preparation and clinical evaluation of a novel lozenge containing polaprezinc, a zinc-L-carnosine, for prevention of oral mucositis in patients with hematological cancer who received high-dose chemotherapy. Med Oncol. 2016; 33(8): 91. doi:10.1007/s12032-016-0795-z.

7. Hewlings S, Kalman D. A Review of Zinc-L-Carnosine and Its Positive Effects on Oral Mucositis, Taste Disorders, and Gastrointestinal Disorders. Nutrients. 2020;12(3):665. Published 2020 Feb 29. doi:10.3390/nu12030665.

8. Janc JW, Clark JM, Warne RL, Elrod KC, Katz BA, Moore WR. A novel approach to serine protease inhibition: kinetic characterization of inhibitors whose potencies and selectivities are dramatically enhanced by Zinc(II). Biochemistry. 2000; 39(16): 4792-4800.

9. Jindal C, Kumar S, Sharma S, Choi YM, Efird JT. The prevention and management of COVID-19: Seeking a practical and timely solution. Int J Environ Res Public Health 2020: 17: 3986; doi:10.3390/ijerph1713986.

10. Kawahara M, Tanaka KI, Kato-Negishi M. Zinc, Carnosine, and Neurodegenerative Diseases. Nutrients. 2018; 10(2): 147. Published 2018 Jan 29. doi:10.3390/nu10020147.

11. Krężel A, Maret W. The biological inorganic chemistry of zinc ions. Arch Biochem Biophys. 2016; 611: 3-19.

12. Kulik L, Maywald M, Kloubert V, Wessels I, Rink L. Zinc deficiency drives Th17 polarization and promotes loss of Treg cell function. J Nutr Biochem. 2019; 63: 11-18.

13. Masafumi Sakagami, Minoru Ikeda, Hiroshi Tomita, Akihiro Ikui, Tsunemasa Aiba, Noriaki Takeda, Akira Inokuchi, Yuichi Kurono, Mitsuyoshi Nakashima, Yuji Shibasaki & Osamu Yotsuya (2009) A zinc-containing compound, Polaprezinc, is effective for patients with taste disorders: randomized, double-blind, placebo-controlled, multi-center study, Acta Oto-Laryngologica, 129:10, 1115-1120.

14. Matsukura T, Tanaka H. Applicability of zinc complex of L-carnosine for medical use. Biochemistry (Mosc). 2000; 65(7): 817-823.

15. Maywald M, Wessels I, Rink L. Zinc Signals and Immunity. Int J Mol Sci. 2017; 18(10): 2222. Published 2017 Oct 24. doi:10.3390/ijms18102222.

16. Prokopieva VD, Yarygina EG, Bokhan NA, Ivanova SA. Use of Carnosine for Oxidative Stress Reduction in Different Pathologies. OxidMed Cell Longev. 2016; 2016: 2939087. doi:10.1155/2016/2939087.

17. Read SA, Obeid S, Ahlenstiel C, Ahlenstiel G. The Role of Zinc in Antiviral Immunity. AdvNutr. 2019;10(4):696-710. doi:10.1093/advances/nmz013

18. Reddy VP, Garrett MR, Perry G, Smith MA. Carnosine: a versatile antioxidant and antiglycating agent. Sci Aging Knowledge Environ. 2005 May 4;2005(18):pe12. doi: 10.1126/sageke.2005.18.pe12.

19. Rosenkranz E, Metz CH, Maywald M, et al. Zinc supplementation induces regulatory T cells by inhibition of Sirt-1 deacetylase in mixed lymphocyte cultures. Mol Nutr Food Res. 2016; 60(3): 661-671.

20. Shang J, Wan Y, Luo C, et al. Cell entry mechanisms of SARS-CoV-2. Proc Natl Acad Sci USA. 2020; 117(21): 11727-11734.

21. te Velthuis AJ, van den Worm SH, Sims AC, Baric RS, Snijder EJ, van Hemert MJ. Zn(2+) inhibits coronavirus and arterivirus RNA polymerase activity in vitro and zinc ionophores block the replication of these viruses in cell culture. PLoS Pathog. 2010 Nov 4; 6(11): e1001176. doi: 10.1371/journal.ppat.1001176.

22. Wessels I, Maywald M, Rink L. Zinc as a Gatekeeper of Immune Function. Nutrients. 2017; 9(12): 1286. Published 2017 Nov 25. doi:10.3390/nu9121286.

All publications and patents mentioned in the above specification are herein incorporated by reference. Various modifications and variations of the described method of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in medicine, medicinal chemistry, organic chemistry, virology, biology, genetics, or related fields are intended to be within the scope of the following claims.

We claim:

1. A method for preventing, inhibiting or treating viral infections or their physiological immune-related sequellae caused by single-stranded RNA viruses in a subject in need thereof comprising administering parenterally to the subject an amount of chelate of zinc and L-carnosine effective to treat or to prevent infections associated with said single-stranded viruses in cells in said subject.

2. The method of claim 1, wherein said chelate of zinc and L-carnosine is first admixed with a carrier in the amount of 1 mg to 41 mg zinc L-carnosine chelate per milliliter or per gram of carrier.

3. The method of claim 1, wherein the subject is a human subject.

4. The method of claim 1, wherein the single-stranded RNA virus is a coronavirus.

5. The method of claim 4, wherein the coronavirus is a SARS-CoV virus.

6. The method of claim 5, wherein the SARS-CoV virus is SARS-CoV-19.

7. The method of claim 1, wherein the chelate of zinc and L-camitine further comprises a second zinc ionophore.

8. The method of claim 7, wherein the second zinc ionophore is selected from the group consisting of L-histidine, L-anserine, L-cysteine, penicillamine, and 2-picolinic acid.

9-16. (canceled)

17. A kit comprising a first container comprising an effective dose of chelate of zinc and L-carnosine and a second container comprising a sterile diluent.

18-22. (canceled)

23. Kit of claim 17, wherein the chelate of zinc and L-camitine further comprises a second zinc ionophore.

24. Kit of claim 23, wherein the second zinc ionophore is selected from the group consisting of L-histidine, L-anserine, L-cysteine, penicillamine, and 2-picolinic acid.
